(19) United States
(12) Patent Application Publication
(10) Pub. No.: US 2025/0149110 A1
(43) Pub. Date: May 8, 2025

(54) METHOD AND APPARATUS FOR PREDICTING STRUCTURE OF PROTEIN COMPLEX

(71) Applicant: BEIJING BAIDU NETCOM SCIENCE TECHNOLOGY CO., LTD., Beijing (CN)

(72) Inventors: Kunrui Zhu, Beijing (CN); Lihang Liu, Beijing (CN); Xiaomin Fang, Beijing (CN); Xiaonan Zhang, Beijing (CN); Jingzhou He, Beijing (CN)

(73) Assignee: BEIJING BAIDU NETCOM SCIENCE TECHNOLOGY CO., LTD., Beijing (CN)

(21) Appl. No.: 18/929,408

(22) Filed: Oct. 28, 2024

(30) Foreign Application Priority Data
Nov. 8, 2023 (CN) 202311477801.2

(51) Int. Cl. G16B 15/20; G16B 40/00

(52) U.S. Cl. CPC G16B 15/20; G16B 40/00

(57) ABSTRACT

A method for predicting a structure of a protein complex includes: obtaining an initial coordinate of each amino acid residue in a target protein complex, and obtaining a target residue pair feature, a first multiple sequence alignment (MSA) feature and a second MSA feature of each protein monomer in the target protein complex; and inputting the initial coordinate of each amino acid residue, and the target residue pair feature, the first MSA feature and the second MSA feature of each protein monomer into an N-level fold iteration network layer, and obtaining a target coordinate of each amino acid residue by predicting a torsion angle, a position transformation at residue level and a position transformation at monomer chain level of each amino acid residue via the N level fold iteration network layer, to obtain a predicted structure of the protein complex.

### CROSS-REFERENCE TO RELATED APPLICATION

This application claims priority to Chinese Patent Application No. 202311477801.2, filed on Nov. 8, 2023, the entire content of which is incorporated herein by reference.

### TECHNICAL FIELD

The disclosure relates to a field of artificial intelligence technologies, more particularly to a field of natural language processing technologies, biological computing technologies, and the like.

### BACKGROUND

A protein complex is a stable macromolecular complex formed by interaction of two or more protein molecules, and the protein complex plays an important role in different biological functions, such as enzyme reaction, cell signaling, metabolic regulation and gene expression. To a great extent, a function of a protein is determined by its own spatial structure. A technology of predicting a spatial three-dimensional structure (tertiary structure) of the protein based on a type of an amino acid (primary structure) of a protein chain is of great research value in a field of life science.

Therefore, it becomes one of important research directions how to accurately predict a structure of the protein and to improve an efficiency of predicting a structure of the protein complex for meeting various biological applications.

### SUMMARY

According to a first aspect of the disclosure, a method for predicting a structure of a protein complex is provided. The method includes:
obtaining an initial coordinate of each amino acid residue in a target protein complex, and obtaining a target residue pair feature, a first multiple sequence alignment (MSA) feature and a second MSA feature of each protein monomer in the target protein complex; and
inputting the initial coordinate of each amino acid residue, and the target residue pair feature, the first MSA feature and the second MSA feature of each protein monomer to an N-level fold iteration network layer, obtaining a target coordinate of each amino acid residue by predicting a torsion angle, a position transformation at residue level and a position transformation at monomer chain level of each amino acid residue via the N-level fold iteration network layer, and obtaining a predicted structure of the protein complex based on the target coordinate of each amino acid residue.

The first MSA feature is an MSA feature subjected to normalization, the second MSA feature is an MSA feature subjected to a mapping process, and N is an integer greater than 1.

According to a second aspect of the disclosure, an electronic device is provided. The electronic device includes: at least one processor, and
a memory communicatively coupled to the at least one processor.

The memory is configured to store instructions executable by the at least one processor. When the instructions are executed by the at least one processor, the at least one processor is configured to realize the method for predicting the structure of the protein complex in the first aspect of embodiments of the disclosure.

According to a third aspect of the disclosure, a non-transitory computer-readable storage medium for storing computer instructions is provided. When the computer instructions are executed by a computer, the method for predicting the structure of the protein complex in the first aspect of embodiments of the disclosure is realized.

### BRIEF DESCRIPTION OF THE DRAWINGS

The accompanying drawings are used to better understand this solution and do not constitute a limitation to the disclosure.

FIG. 1 is a flow chart illustrating a method for predicting a structure of a protein complex according to an embodiment of the disclosure.

FIG. 2 is a flow chart illustrating a method for predicting a structure of a protein complex according to an embodiment of the disclosure.

FIG. 3 is a schematic diagram illustrating a method for predicting a structure of a protein complex according to an embodiment of the disclosure.

FIG. 4 is a flowchart illustrating a method for predicting a structure of a protein complex according to an embodiment of the disclosure.

FIG. 5 is a schematic diagram schematic a method for predicting a structure of a protein complex according to an embodiment of the disclosure.

FIG. 6 is a block diagram illustrating an apparatus for predicting a structure of a protein complex according to an embodiment of the disclosure.

FIG. 7 is a block diagram illustrating an electronic device used to implement the method according to an embodiment of the disclosure.

### DETAILED DESCRIPTION

Description is made below to exemplary embodiments of the disclosure with reference to the accompanying drawings, which includes various details of embodiments of the disclosure to aid in understanding, and should be considered merely exemplary. Those skilled in the art should understand that various changes and modifications of embodiments described herein may be made without departing from the scope and spirit of the disclosure. For the sake of clarity and brevity, descriptions of well-known functions and structures are omitted from the following description.

Embodiments of the disclosure relate to a field of artificial intelligence technologies, such as computer vision, deep learning, and the like.

Artificial intelligence (AI) is a new technological science that studies and develops theories, methods, technologies and application systems for simulating, extending and expanding human intelligence.

Natural language processing (NLP) is an important direction in the fields of computer science and AI. The NLP studies various theories and methods that realize effective communication in natural language between humans and computers. The NLP is a science that integrates linguistics, computer science, and mathematics. Therefore, researches in the field involve natural language, i.e., daily language used by people, so the NLP is closely related to the study of linguistics, but with great differences. However, the NLP is not the study of natural language in general, but the NLP mainly focuses on developing a computer system that may effectively realize communications in natural language, especially in a software system. Therefore, it means that the NLP also belongs to a part of computer science.

Biological computing refers to a new computing way studied and developed by utilizing inherent information processing mechanisms in a biological system. The biological computing mainly studies two aspects, i.e., a device and a system. The biological computing utilizes organic (or biological) materials to constitute an ordered system at a molecular scale, and to provide a basic unit for detecting, processing, transmitting and storing information through physicochemical processes at a molecular level.

Description is made below to a method and apparatus for predicting a structure of a protein complex with reference to the accompany drawings.

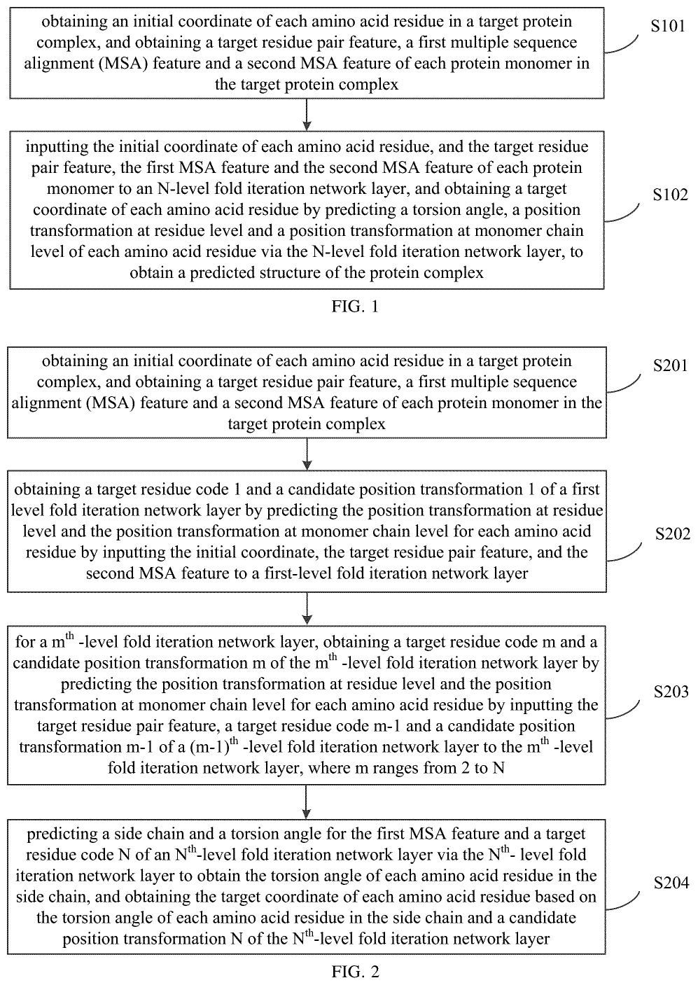

FIG. 1 is a flow chart illustrating a method for predicting a structure of a protein complex according to an embodiment of the disclosure. As illustrated in FIG. 1, the method includes the following.

At block S101, an initial coordinate of each amino acid residue in a target protein complex is obtained, and a target residue pair feature, a first multiple sequence alignment (MSA) feature and a second MSA feature of each protein monomer in the target protein complex are obtained.

The protein complex has multiple protein monomers, and each protein monomer has only one amino-acid sequence. Amino acids lose a molecule of water when the amino acids bind to each other to form a peptide bond, thus an amino acid unit in polypeptide/protein is referred to as amino acid residue. In embodiments of the disclosure, in order to accommodate a rotational invariance of the protein structure, relative position transformation is employed to represent a coordinate of each residue, and a spatial structure of the protein complex is initialized with a coordinate origin. That is, a coordinate of each amino acid residue in the target protein complex is initialized to obtain an initial coordinate Tᵢ=(I,\vec{0}), where Tᵢ=(I,\vec{0}) represents a coordinate origin in a way of rotation/translation, I is a unit matrix and represents no rotation, \vec{0} vector represents no translation, and i represents an ith amino acid residue.

In embodiments of the disclosure, a template feature of each protein monomer is obtained, and a pairing feature of an amino acid sequence of each protein monomer is constructed. For each protein monomer, the target residue pair feature of the protein monomer is obtained based on the template feature and the pairing feature of the protein monomer.

In some implementations, for each protein monomer, a homologous sequence of the protein monomer is obtained by querying multiple gene sequence data bases based on the target amino acid sequence of the protein monomer. Multiple sequence alignment is performed on the homologous sequence of the protein monomer to obtain an MSA feature of the protein monomer. Different processes are performed on the MSA feature to obtain a first MSA feature and a second MSA feature. Alternatively, the first MSA feature is an MSA feature subjected to normalization, and the second MSA feature is an MSA feature subjected to mapping process.

At block S102, the initial coordinate of each amino acid residue, and the target residue pair feature, the first MSA feature and the second MSA feature of each protein monomer are inputted to an N-level fold iteration network layer, and a target coordinate of each amino acid residue is obtained by predicting a torsion angle, a position transformation at residue level and a position transformation at monomer chain level of each amino acid residue via the N level fold iteration network layer, to obtain a predicted structure of the protein complex.

N is an integer greater than 1.

Alternatively, a torsion angle in a residual side chain is predicted based on a side chain and torsion angle predictor in the N-level fold iteration network layer.

In some implementations, when the structure of the protein complex is predicted, residue codes of multiple chains in the protein complex are directly mapped to coordinate transformations. These transformations only act on the residues, and such type of transformation in the disclosure is referred to as position transformation at residue level.

In the disclosure, a relative independence of each monomer chain in the protein complex is taken into consideration, and the position transformation at monomer chain level is performed on the basis of the position transformation at residue level to update the coordinate of each amino acid residue, thus realizing decoupling of predicting a position of a residue in a chain and predicting an overall position of a sub chain, and enhancing overall effectiveness of a prediction model for a protein structure.

In embodiments of the disclosure, the initial coordinate of each amino acid residue, and the target residue pair feature, the first MSA feature and the second MSA feature of each protein monomer are input to the N-level fold iteration network layer, and the target coordinate of each amino acid residue may be obtained by predicting the torsion angle, the position transformation at residue level and the position transformation at monomer chain level of each amino acid residue via the N level fold iteration network layer. The relative independence of each monomer chain in the protein complex is taken into consideration, and the position transformation at monomer chain level is performed on the basis of the position transformation at residue level to update the coordinate of each amino acid residue, thus accurately predicting the protein structure, improving the efficiency of predicting the structure of the protein complex, and better adapting to an application scene where the protein complex includes multiple chains.

FIG. 2 is a flow chart illustrating a method for predicting a structure of a protein complex according to an embodiment of the disclosure. As illustrated in FIG. 2, the method includes the following.

At block S201, an initial coordinate of each amino acid residue in a target protein complex is obtained, and a target residue pair feature, a first MSA feature and a second MSA feature of each protein monomer in the target protein complex are obtained.

Description about block S201 may be referred to relevant description in the above embodiments, which is not repeated here.

At block S202, a target residue code 1 and a candidate position transformation 1 of a first-level fold iteration network layer are obtained by predicting the position transformation at residue level and the position transformation at monomer chain level for each amino acid residue by inputting the initial coordinate, the target residue pair feature, and the second MSA feature into the first-level fold iteration network layer.

Residue codes with the rotational invariance are obtained by executing invariant point attention mechanism on the initial coordinate, the target residue pair feature and the second MSA feature via the first-level fold iteration network layer, and then a mapping processing is performed on the residue codes by a linear network to obtain the target residue code 1.

With performing the mapping process on the target residue code of each amino acid residue based on a backbone update algorithm, it is implemented that a first position transformation 1 of each amino acid residue is obtained by performing position transformation prediction at residue-level on the target residue code 1, and a second position transformation 1 of each amino acid residue is obtained by performing position transformation prediction at monomer chain level on the target residue code 1.

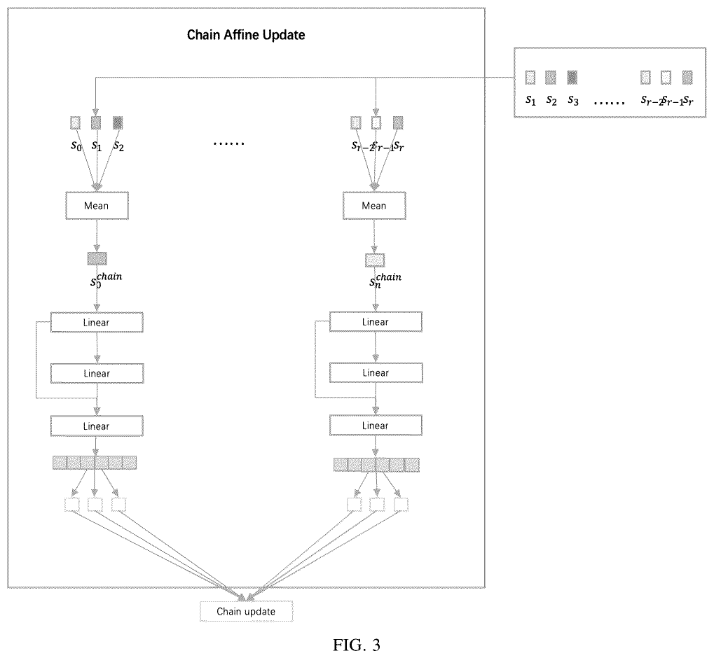

As illustrated in FIG. 3, in some implementations, an update process of the position transformation at monomer chain level (Chain Affine Update) includes: for each amino acid residue, dividing two or more adjacent amino acid residues into different monomer chains based on the target residue code of the amino acid residue. For example, three adjacent amino acid residues are divided into a same monomer chain. That is, for given the residue codes [s1, s2, s3, . . . , si, . . . , sr], a table [1, 2, 3, . . . , i, . . . , r] may be employed to locate a monomer chain before splicing (for example, s1 to s3 belong to a monomer chain 1, and sr−2 to sr belong to a monomer chain n), mean calculation is performed on the target residue codes of each monomer chain to obtain a representation at monomer chain level, i.e., a candidate residue code.

In embodiments of the disclosure, for target amino acid residues on any monomer chain, mean calculation is performed on the target residue codes of the target amino acid residue to obtain the candidate residue code at monomer chain level. A mapping process is performed on the candidate residue code based on a multi-layer neural network structure (e.g., a multi-layer linear network (Linear) illustrated in FIG. 3), to obtain a second position transformation of each amino acid residue in the monomer chain.

As illustrated in FIG. 3, in some implementations, the multi-layer neural network structure includes three layers of linear networks. A first transformation representation is obtained by inputting the candidate residue code to a first linear network in the three layers of linear networks for a mapping process. A second transformation representation is obtained by inputting the first transformation representation to a second linear network in the three layers of linear networks for a mapping process. The second position transformation of each amino acid residue in the monomer chain is obtained by inputting the first transformation representation and the second transformation representation to a third linear network in the three layers of linear networks for a mapping process. The three layers may have a same structure or different structures, which is not limited in embodiments of the disclosure.

The candidate position transformation 1 of the first-level fold iteration network layer is obtained by performing position update based on the first position transformation 1, the second position transformation 1 and the initial coordinate.

At block S203, for a mth-level fold iteration network layer, a target residue code m and a candidate position transformation m of the mth-level fold iteration network layer are obtained by predicting the position transformation at residue level and the position transformation at monomer chain level for each amino acid residue by inputting the target residue pair feature, a target residue code m−1 and a candidate position transformation m−1 of a (m−1)th-level fold iteration network layer to the mth-level fold iteration network layer, where m ranges from 2 to N.

Invariant point attention mechanism is performed on the candidate position transformation (m−1)th and the target residue pair feature of the (m−1)th-level fold iteration network layer inputted to the mth-level fold iteration network layer, to obtain a residue code with the rotational invariance, and then mapping process is performed on the residue code by the linear network to obtain the target residue code m.

Similarly, after employing the action at block S202, the position transformation prediction at residue-level is performed on the target residue code m to obtain a first position transformation m of each amino acid residue, the position transformation prediction at monomer chain level is performed on the target residue code m to obtain a second position transformation m of each amino acid residue, and the candidate position transformation m of the mth-level fold iteration network layer is obtained based on the first position transformation m and the second position transformation m. Based on the same principle, a candidate position transformation N and a target residue code N of an Nth-level fold iteration network layer are obtained.

At step S204, a side chain and a torsion angle is predicted for the first MSA feature and a target residue code N of the Nth-level fold iteration network layer via the Nth-level fold iteration network layer to obtain a torsion angle of each amino acid residue in the side chain, and the target coordinate of the amino acid residue is obtained based on the torsion angle of the amino acid residue in the side chain and the candidate position transformation N of the Nth-level fold iteration network layer.

The first MSA feature and the target residue code N of the Nth-level iterative network layer are input to a side chain and a torsion angle predictor of the Nth-level fold iteration network layer to obtain the torsion angle of the amino acid residue in the side chain. The target coordinate of the amino acid residue is obtained by performing position update based on the torsion angle of each amino acid residue in the side chain, and the candidate position transformation N of the Nth-level fold iteration network layer.

In embodiments of the disclosure, the relative independence of each monomer chain in the protein complex is taken into consideration, and the position transformation at monomer chain level is performed on the basis of the position transformation at residue level, to update the coordinate of each amino acid residue, thus realizing decoupling of predicting the position of the residue in the chain and predicting the overall position of the sub chain, and enhancing the overall effectiveness of the prediction model for the protein structure. In this way, a docking relationship between the chains may be adjusted globally while the relative positions of residues in the single chain is reserved better, more suitable for predicting the structure of the protein complex.

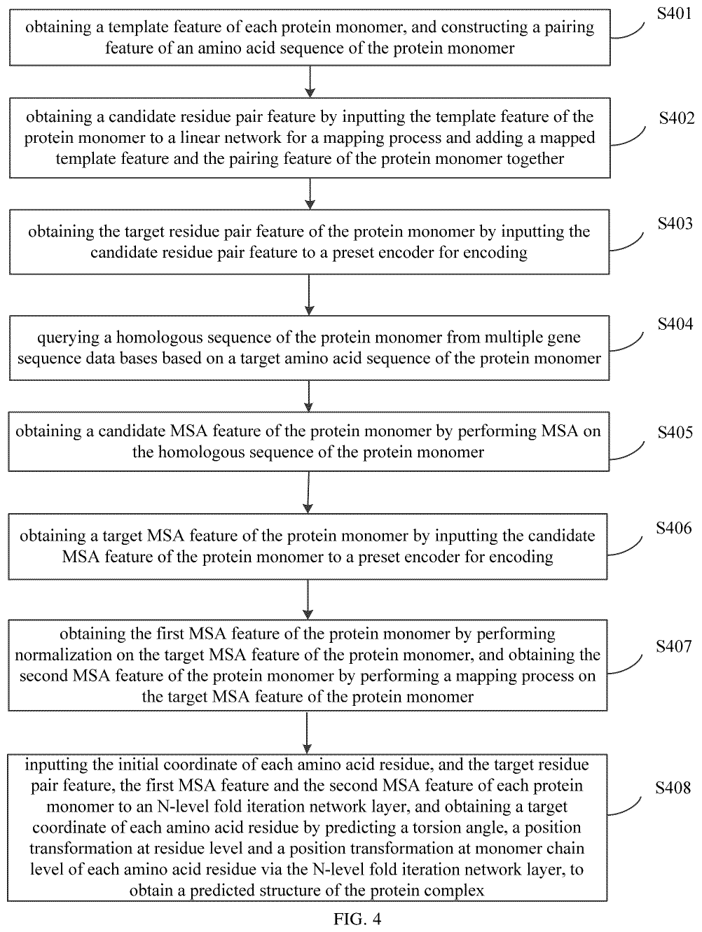

FIG. 4 is a flow chart illustrating a method for predicting a structure of a protein complex according to an embodiment of the disclosure. As illustrated in FIG. 4, the method includes the following.

At block S401, a template feature of each protein monomer is obtained, and a pairing feature of an amino-acid sequence of the protein monomer is constructed.

In some implementations, a target amino acid sequence of each protein monomer is matched with multiple first amino acid sequences in a protein structure data base respectively to obtain a second amino acid sequence with a similarity greater than a preset threshold. A distance between coordinates of amino acid residues in the second amino acid sequence is determined as the template feature of the protein monomer. That is, a protein structure similar to the amino acid sequence of the protein monomer may be determined by querying a data base including analytical protein structures. The distance between the residues is extracted by a tool for analyzing a protein sequence, such as a hidden markov model (HMM) based search method (HHSearch), and the distance is taken as a template feature (Template).

In some implementations, candidate sequence code features are obtained by inputting the amino acid sequence of each protein monomer into two preset linear networks. A first sequence code feature and a second sequence code feature are obtained by adding null dimensions to different directions of the candidate sequence code features respectively. The pairing feature of each protein monomer is obtained by adding the first sequence code feature and the second sequence code feature together. A complex sequence with a length r and spliced by multiple sequences is encoded by the two linear networks (Linear), to obtain a sequence code feature in a shape of [r, c], i.e., a first sequence code feature z₁ and a second sequence code feature z₂, where c represents a depth (which is a hyper-parameter) of hidden layers of the Linear network layer. Therefore, null dimensions are respectively added to z₁ and z₂ (the shape of z₁ is converted to [r,1,c], and the shape of z₂ is converted to [1,r,c]), and z₁ and z₂ are added to obtain a pairing feature zₚₐᵢᵣ, where a shape of zₚₐᵢᵣ is [r, r, c], zₚₐᵢᵣ=z₁+z₂.

At block S402, a candidate residue pair feature is obtained by inputting the template feature of each protein monomer into a linear network for a mapping process and adding a mapped template feature and the pairing feature of the protein monomer together.

In embodiments of the disclosure, the template feature is in the shape of [r, r] after splicing, a feature zₜₑₘₚ with a consistent shape with the paring feature zₚₐᵢᵣ is obtained after encoding via the linear layer, and the feature zₜₑₘₚ is added to the paring feature to obtain the candidate residue pair feature.

At step S403, the target residue pair feature of the protein monomer is obtained by inputting the candidate residue pair feature to a preset encoder for encoding.

In embodiments of the disclosure, the candidate residue pair feature is inputted to the preset encoder (Evofomer Encoder) for encoding to obtain the target residue pair feature of the protein monomer.

At step S404, a homologous sequence of the protein monomer is queried from multiple gene sequence data bases based on a target amino acid sequence of the protein monomer.

In the disclosure, firstly, the amino-acid sequence of each protein monomer in the complex is taken as a query request to query the homologous sequence in the multiple gene sequence data bases. More in-depth analysis and annotation of the protein sequence may be achieved using an existing tool, such as JackHMMER and HHblits. The JackHMMER is employed for fast heuristic query of the HMM, and the HHblits is employed for more in-depth annotation of a discovered protein sequence, thus obtaining the homologous sequence for each protein monomer.

At block S405, a candidate MSA feature of the protein monomer is obtained by performing MSA on the homologous sequence of the protein monomer.

MSA is performed on an obtained homologous sequence to obtain an MSA (MSA) feature for each protein monomer.

At block S406, a target MSA feature of the protein monomer is obtained by inputting the candidate MSA feature of the protein monomer to a preset encoder for encoding.

The candidate MSA feature of each protein monomer is encoded by the encoder (Evofomer Encoder) to obtain the target MSA feature of the protein monomer.

At block S407, the first MSA feature of the protein monomer is obtained by performing normalization on the target MSA feature of the protein monomer, and the second MSA feature of the protein monomer is obtained by performing a mapping process on the target MSA feature of the protein monomer.

Normalization is performed on the target MSA feature of each protein monomer to obtain the first MSA feature of the protein monomer. Mapping processing is performed on the target MSA feature of each protein monomer based on the linear network to obtain the second MSA feature of the protein monomer.

At block S408, the initial coordinate of each amino acid residue, and the target residue pair feature, the first MSA feature and the second MSA feature of the protein monomer are input to an N-level fold iteration network layer, and a target coordinate of each amino acid residue is obtained by predicting a torsion angle, a position transformation at residue level and a position transformation at monomer chain level of each amino acid residue via the N-level fold iteration network layer, to obtain a predicted structure of the protein complex.

Description about block S408 may be referred to the relevant content in above embodiments, which is not repeated here.

In embodiments of the disclosure, the initial coordinate of each amino acid residue in the target protein complex, and the target residue pair feature, the first MSA feature, and the second MSA feature of each protein monomer in the target protein complex are obtained, thus greatly facilitating a development of a prediction task of a protein monomer structure, and enhancing the overall effectiveness of predicting the protein structure.

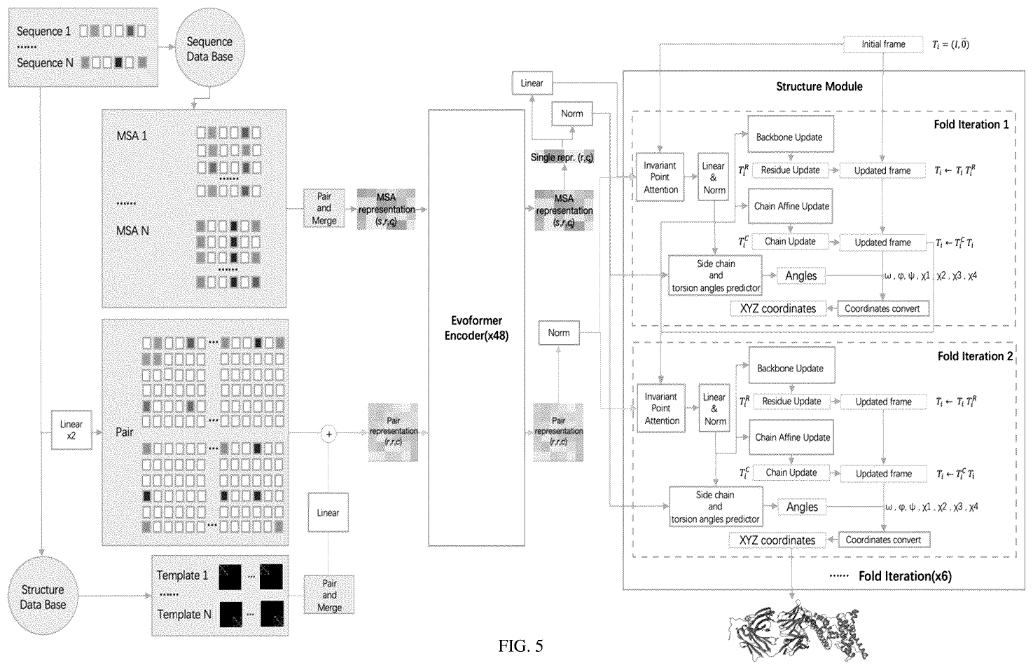

FIG. 5 is a schematic diagram illustrating a method for predicting a structure of a protein complex according to an embodiment of the disclosure. As illustrated in FIG. 5, in embodiments of the disclosure, to query the homologous sequence, the multiple gene sequence data bases are queried based on the amino acid sequence (i.e., [Sequence 1, . . . , Sequence N]) of each protein monomer in the target protein complex, and then MSA is performed to obtain the MSA feature (i.e., [MSA 1, . . . , MSA N]) of each protein monomer. The MSA feature (i.e., [MSA 1, . . . , MSA N]) is inputted to the Evofomer encoder for encoding to obtain the target MSA feature. The amino acid sequence of each protein monomer is input into two preset linear networks (Linear) to generate the pairing feature. Sequences in the protein structure data base are queried based on the target amino-acid sequence of each protein monomer to obtain a similar protein structure, and a distance between the residues is extracted as a template feature. Alternatively, “Pair and Merge” represents merging. That is, an MSA representation feature and a pair representation feature are merged and processed. The template feature of each protein monomer is inputted to the linear network for a mapping processing, and a mapped template feature is added to the pairing feature of the protein monomer and inputted to the Evofomer encoder for encoding, to obtain the target residue pair feature of the protein monomer. The Evofomer encoder may extract an implicit code of each residue from MSA data, pair data and template data at a decoding stage. In the disclosure, a training task may be assisted based on a structure prediction module in a structure prediction model (AF2Multimer) of the protein complex, MSA mask prediction, local distance different test (LDDT) prediction (LDDT is a pre-existing metric way in a field of predicting the protein structure), and residue distance prediction. An initial frame represents an initial coordinate.

As illustrated in FIG. 5, a protein complex including r residues is input into the Evofomer encoder for encoding, to obtain an MSA feature code of each ith residue of the target protein complex, i.e., the target MSA feature, Sᵢ^{initial} ∈R^{1×c}^{ₛ}, i∈[0, 1, . . . , r] (where cₛ represents a size of a hyper-parameter hidden layer), and a residue pair code, i.e., Z_{i,j}∈R^{1×c}, i, j∈[0, 1, . . . , r], including the pairing feature and the template feature. The target MSA feature of each protein monomer is inputted into a normalization layer (Norm) in the network to obtain the first MSA feature of each protein monomer, and the target MSA feature of each protein monomer is inputted into a linear network (Linear) to obtain the second MSA feature of each protein monomer. R represents the shape, that is, represents that a shape of sᵢ^{initial} is [1,c], and c is the depth of the hidden layers of the model.

In embodiments of the disclosure, the structure of the target protein complex is predicted by employing the N-level fold iteration network layer (fold iteration module). Alternatively, N may take a value of 8. In other implementations, N may take other values, which is not limited in embodiments of the disclosure.

In the disclosure, in order to adapt to the rotational invariance of the protein structure, the relative position transformation Tᵢ=(Rᵢ, \vec{t₁}) is employed to represent the coordinate of each residue, and the spatial structure of the protein complex is initialized with a coordinate origin Tᵢ=(1,\vec{0}). In the disclosure, first, Sᵢ^{initial} and Z_{i,j} codes are updated with two Norm layers in the network, and the Sᵢ^{initial} code is mapped to a hidden layer representation sᵢ by employing the linear layer (Linear), where sᵢ represents a code of an ith residue, Z_{i,j} represents pairing codes of the ith residue to the jth residue. Tᵢ represents rotation and translation of the ith residue. Rᵢ represents a rotational transformation of the ith residue, and t; represents a shift transformation of the ith residue. An absolute coordinate is converted into a relative rotation and translation to represent the residue coordinate based on an Alphafold model to realize the rotational invariance.

After sᵢ, Z_{i,j}, and Tᵢ are accessed, each layer of Fold Iterations obtains the residue code sᵢ with the rotational invariance by employing an invariant point attention module. After the residue code sᵢ passes through the network layers such as Linear, Norm, and Dropout layers to obtain a code, in the disclosure, an obtained code is employed to predict the torsion angle aᵢ^{f}∈R² of each residue in the side chain and the coordinate Tₖ^{C}=(Rₖ^{C}, tₖ^{C}) of each residue. The Dropout layer is employed to randomly discard a network parameter, which plays a less role and is not illustrated in FIG. 5, thus the layer may be omitted or deleted.

As illustrated in FIG. 5, in embodiments of the disclosure, a side chain and a torsion angle predictor in each layer of Fold Iterations are employed to predict a torsion angle aᵢ^{f}∈R² of the ith residue in the side chain, where f∈{ωϕψχ₁χ₂χ₃χ₄} represents 7 components that may be twisted in the side chain of each residue.

In the disclosure, for prediction of a backbone network structure for the protein complex, the residue feature is encoded first by employing a shallow neural network structure (Linear and Norm), and then an Euclidean transformation Tᵢ of the ith residue is predicted by a BackboneUpdate algorithm, i.e., the first position transformation. In the BackboneUpdate algorithm, the hidden layer feature is mapped to a 6-dimensional representation, where the first three dimensions bᵢ, cᵢ, and dᵢ are used as a rotation matrix Rᵢ of the ith residue after the equation, and the last three dimensions \vec{t₁} directly represent the shift transformation of the ith residue. The inputs to the Fold Iterations do not take into account the side chain and the torsion angle predicted at the previous step, but only the position transformation (affine) of a main chain and the residue code.

After a spatial position transformation prediction Tᵢ^{R}=(Rᵢ,\vec{tᵢ}) of the ith residue is obtained, the model updates the relative position of the ith residue based on Tᵢ=Tᵢ∘Tᵢ^{R}, to complete the position transformation at residue level. In the equation, the first Tᵢ represents the updated Tᵢ, and the second Tᵢ represents the Tᵢ before update.

In the disclosure, on the basis of the above position transformation, the position transformation module at monomer chain level (Chainaffine) is introduced to predict the position transformation at monomer chain level on the residue. The Chainaffine module aims at predicting an overall transformation Tₖ^{C} of a protein complex chain k, and a module structure of the Chainaffine module may be realized in multiple ways. FIG. 3 illustrates one realizing method of the Chainaffine module. Taking; as input, the Chainaffine module first divides sᵢ into different monomer chains based on a code of the position of the residue, and obtains a hidden layer representation sₖ^{chain}∈R^{1×d}, k∈[0, 1, . . . , n] (where, n represents the number of sub chains of the protein complex, and d represents a size of a hidden layer of a Chainaffine network) at monomer chain level after calculating a mean value of all residue representations of the same monomer.

Then, the Chainaffine module maps Sₖ^{chain} to a transformation representation including 6 dimensions by employing the multi-layer neural network structure, and obtains a spatial position transformation, i.e., the second position transformation, of each chain by employing a same method as the BackboneUpdate.

As illustrated in FIG. 5, when the position of the residue in the chain is updated, all residues on the chain k share the transformation Tₖ^{C} to obtain the second position transformation Tᵢ^{C} at monomer chain level of each residue. Finally, the model updates the position of each residue in the protein complex based on Tᵢ=Tᵢ∘Tᵢ^{C}, where, the first Tᵢ represents the updated Tᵢ, the second Tᵢ represents the Tᵢ before update, and Tᵢ^{C} acts on the front of Tᵢ to convert the residues in the chain around the origin.

In embodiments of the disclosure, after the Euclidean transformation of the backbone network and the torsion angle of the side chain are obtained, Tᵢ and aᵢ^{f} are converted into a three-dimensional coordinate of each residue based on a residue update module and an update frame module, to complete tertiary structure prediction on the protein. An angle module is configured to predict the torsion angle of the side chain, and a coordinate convert module is configured to output a converted spatial coordinate after receiving the main chain transformation and the torsion angle of the side chain.

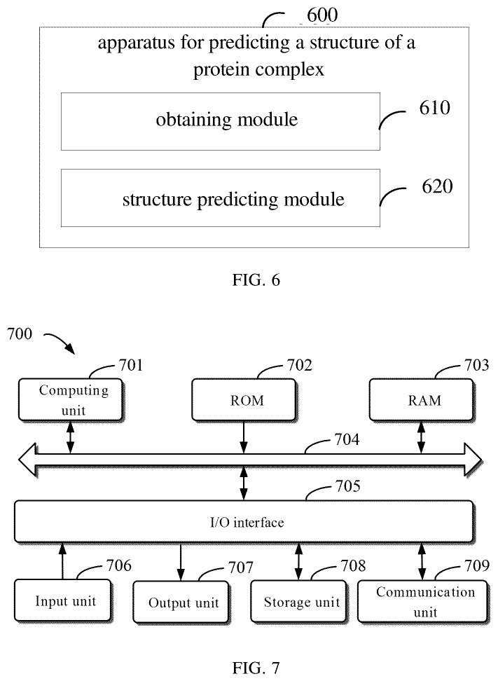

FIG. 6 is a block diagram illustrating an apparatus for predicting a structure of a protein complex according to an embodiment of the disclosure. As illustrated in FIG. 6, the apparatus 600 includes:
an obtaining module 610, configured to obtain an initial coordinate of each amino acid residue in target a protein complex, and to obtain a target residue pair feature, a first multiple sequence alignment (MSA) feature and a second MSA feature of each protein monomer in the target protein complex; and
a structure predicting module 620, configured to input the initial coordinate of each amino acid residue, and the target residue pair feature, the first MSA feature and the second MSA feature of each protein monomer to an N-level fold iteration network layer, and to obtain a target coordinate of each amino acid residue by predicting a torsion angle, a position transformation at residue level and a position transformation at monomer chain level of each amino acid residue via the N-level fold iteration network layer, to obtain a predicted structure of the protein complex.

The first MSA feature is an MSA feature subjected to normalization, the second MSA feature is an MSA feature subjected to a mapping process, and N is an integer greater than 1.

In some implementations, the structure predicting module 620 is further configured to:
obtain a target residue code 1 and a candidate position transformation 1 of a first level fold iteration network layer by predicting the position transformation at residue level and the position transformation at monomer chain level for each amino acid residue by inputting the initial coordinate, the target residue pair feature, and the second MSA feature to a first-level fold iteration network layer;
for a mth-level fold iteration network layer, obtain a target residue code m and a candidate position transformation m of the mth-level fold iteration network layer by predicting the position transformation at residue level and the position transformation at monomer chain level for each amino acid residue by inputting the target residue pair feature, a target residue code m−1 and a candidate position transformation m−1 of a (m−1)th-level fold iteration network layer to the mth-level fold iteration network layer, where m ranges from 2 to N; and
predict a side chain and a torsion angle for the first MSA feature and a target residue code N of an Nth-level fold iteration network layer via the Nth-level fold iteration network layer to obtain the torsion angle of each amino acid residue in the side chain, and obtain the target coordinate of each amino acid residue based on the torsion angle of each amino acid residue in the side chain and a candidate position transformation N of the Nth-level fold iteration network layer.

In some implementations, the structure predicting module 620 is further configured to:
obtain the target residue code 1 by performing invariant point attention mechanism and a mapping process on the initial coordinate, the target residue pair feature, and the second MSA feature via the first-level fold iteration network layer;
obtain a first position transformation 1 of the amino acid residue by performing position transformation prediction at residue level on the target residue code 1, and obtain a second position transformation 1 of the amino acid residue by performing position transformation prediction at monomer chain level on the target residue code 1; and
obtain the candidate position transformation 1 of the first-level fold iteration network layer by performing position update based on the first position transformation 1, the second position transformation 1 and the initial coordinate.

In some implementations, the structure predicting module 620 is further configured to:
obtain the target residue code m by performing invariant point attention mechanism and a mapping process on the candidate position transformation m−1 and the target residue pair feature of the (m−1)th-level fold iteration network layer inputted to the mth-level fold iteration network layer;
obtain a first position transformation m of the amino acid residue by performing position transformation prediction at residue level on the target residue code m, and obtain a second position transformation m of the amino acid residue by performing position transformation prediction at monomer chain level on the target residue code m; and
obtain the candidate position transformation m of the mth-level fold iteration network layer based on the first position transformation m and the second position transformation m.

In some implementations, the structure predicting module 620 is further configured to:
obtain the first position transformation of each amino acid residue by performing a mapping processing on the target residue code of each amino acid residue based on a backbone update algorithm.

In some implementations, the structure predicting module 620 is further configured to:
divide two or more adjacent amino acid residues into different monomer chains based on the target residue code of each amino acid residue; and
for target amino acid residues in each monomer chain, obtain a candidate residue code at monomer chain level by performing a mean calculation on target residue codes of the target amino acid residues, and obtain the second position transformation of each amino acid residue in the monomer chain by performing a mapping process on the candidate residue code based on a multi-layer neural network structure.

In some implementations, the multi-layer neural network structure includes three layers of linear networks, and the structure predicting module 620 is further configured to:
obtain a first transformation representation by inputting the candidate residue code to a first linear network in the three layers of linear networks for a mapping process;
obtain a second transformation representation by inputting the first transform representation in the three layers of linear networks to a second linear network for a mapping process; and
obtain the second position transformation of the amino acid residue in the monomer chain by inputting the first transformation representation and the second transformation representation to a third linear network in the three layers of linear networks for a mapping process.

In some implementations, the obtaining module 610 is further configured to:
obtain a template feature of the protein monomer, and construct a pairing feature of an amino acid sequence of the protein monomer;
obtain a candidate residue pair feature by inputting the template feature of the protein monomer to a linear network for a mapping process and adding a mapped template feature and the pairing feature of the protein monomer together; and
obtain the target residue pair feature of the protein monomer by inputting the candidate residue pair feature to a preset encoder for encoding.

In some implementations, the obtaining module 610 is further configured to:
match a target amino acid sequence of the protein monomer with multiple first amino acid sequences in a protein structure data base respectively to obtain a second amino acid sequence with a similarity greater than a preset threshold; and
determine a distance between coordinates of amino acid residues of the second amino-acid sequence as the template feature of the protein monomer.

In some implementations, the obtaining module 610 is further configured to:
obtain candidate sequence code features by inputting the amino acid sequence of the protein monomer into two preset linear networks;
obtain a first sequence code feature and a second sequence code feature by adding null dimensions to different directions of the candidate sequence code features respectively; and
obtain the pairing feature of the protein monomer by adding the first sequence code feature and the second sequence code feature.

In some implementations, the obtaining module 610 is further configured to:
query a homologous sequence of the protein monomer from multiple gene sequence data bases based on a target amino acid sequence of the protein monomer;
obtain a candidate MSA feature of the protein monomer by performing MSA on the homologous sequence of the protein monomer;
obtain a target MSA feature of the protein monomer by inputting the candidate MSA feature of the protein monomer to a preset encoder for encoding; and
obtain the first MSA feature of the protein monomer by performing normalization on the target MSA feature of the protein monomer, and obtain the second MSA feature of the protein monomer by performing a mapping process on the target MSA feature of the protein monomer.

With the disclosure, the relative independence of each monomer chain in the protein complex is taken into consideration, and the position transformation at monomer chain level is performed on the basis of the position transformation at residue level to update the coordinate of each amino acid residue, thus accurately predicting the protein structure, improving the efficiency of predicting the structure of the protein complex, and better adapting to any application scene where the protein complex includes multiple chains.

Embodiments of the disclosure also provide an electronic device, a readable storage medium, and a computer program product.

FIG. 7 is a block diagram illustrating an electronic device according to an embodiment of the disclosure. The electronic device is configured to realize the method for predicting a structure of the protein complex according to embodiments of the disclosure. The electronic device is intended to represent various forms of digital computers, such as a laptop computer, a desktop computer, a workbench, a personal digital assistant, a server, a blade server, a mainframe computer, and other suitable computer. The electronic device may also represent various forms of mobile devices, such as a personal digital processor, a cellular phone, a smart phone, a wearable device, and other similar computing device. The components illustrated here, their connections and relations, and their functions are merely examples, and are not intended to limit the implementations of the disclosure described and/or required herein.

As illustrated in FIG. 7, the device 700 includes a computing unit 701 for performing various appropriate actions and processes based on computer programs stored in a read-only memory (ROM) 702 or computer programs loaded from a storage unit 708 to a random access memory (RAM) 703. In the RAM 703, various programs and data required for the operation of the device 700 are stored. The computing unit 701, the ROM 702, and the RAM 703 are connected to each other through a bus 704. An input/output (I/O) interface 705 is also connected to the bus 704.

Components in the device 700 are connected to the I/O interface 705, including: an input unit 706, such as a keyboard, a mouse; an output unit 707, such as various types of displays, speakers; a storage unit 708, such as a disk, an optical disk; and a communication unit 709, such as a network card, a modem, and a wireless communication transceiver. The communication unit 709 allows the device 700 to exchange information/data with other devices through a computer network such as the Internet and/or various telecommunication networks.

The computing unit 701 may be various general-purpose and/or dedicated processing components with processing and computing capabilities. Some examples of the computing unit 701 include, but are not limited to, a central processing unit (CPU), a graphics processing unit (GPU), various dedicated AI computing chips, various computing units that run machine learning model algorithms, and a digital signal processor (DSP), and any appropriate processor, controller and microcontroller. The computing unit 701 executes the various methods and processes described above, such as the method for predicting the structure of the protein complex. For example, in some embodiments, the method for predicting the structure of the protein complex may be realized as computer software programs, which are tangibly included in a machine-readable medium, such as the storage unit 708. In some embodiments, part or all of the computer programs may be loaded and/or installed on the device 700 via the ROM 702 and/or the communication unit 709. When the computer programs are loaded on the RAM 703 and executed by the computing unit 701, one or more steps of the method described above may be executed. Alternatively, in other embodiments, the computing unit 701 may be configured to execute the method for predicting the structure of the protein complex in any other suitable manner (for example, by means of firmware).

Various implementations of the systems and techniques described above in the disclosure may be implemented by a digital electronic circuit system, an integrated circuit system, field programmable gate arrays (FPGAs), application specific integrated circuits (ASICs), application specific standard products (ASSPs), system on chips (SOCs), complex programmable logic devices (CPLDs), computer hardware, firmware, software, and/or a combination thereof. These various embodiments may be implemented in one or more computer programs, the one or more computer programs may be executed and/or interpreted on a programmable system including at least one programmable processor. The programmable processor may be a dedicated or general programmable processor for receiving data and instructions from the storage system, at least one input device and at least one output device, and transmitting the data and instructions to the storage system, the at least one input device and the at least one output device.

The program codes configured to realize the method of the disclosure may be written in any combination of one or more programming languages. These program codes may be provided to the processors or controllers of general-purpose computers, dedicated computers, or other programmable data processing devices, such that the program codes, when executed by the processors or controllers, enable the functions/operations specified in the flowchart and/or block diagram to be implemented. The program codes may be executed entirely on the machine, partly executed on the machine, partly executed on the machine and partly executed on the remote machine as an independent software package, or entirely executed on the remote machine or server.

In the context of the disclosure, a machine-readable medium may be a tangible medium that may include or store a program for use by or in combination with an instruction execution system, apparatus, or device. The machine-readable medium may be a machine-readable signal medium or a machine-readable storage medium. The machine-readable medium may include, but is not limited to, an electronic, magnetic, optical, electromagnetic, infrared, or semiconductor system, apparatus, or device, or any suitable combination of the foregoing. More specific examples of the machine-readable storage medium include electrical connections based on one or more wires, portable computer disks, hard disks, RAMs, ROMs, electrically programmable read-only-memories (EPROMs), flash memories, fiber optics, compact disc read-only memories (CD-ROMs), optical storage devices, magnetic storage devices, or any suitable combination of the foregoing.

In order to provide interaction with a user, the systems and techniques described herein may be implemented on a computer having a display device (e.g., a cathode ray tube (CRT) or a liquid crystal display (LCD) monitor for displaying information to a user); and a keyboard and pointing device (such as a mouse or trackball) through which the user can provide input to the computer. Other types of devices may also be configured to provide interaction with the user. For example, the feedback provided to the user may be any form of sensory feedback (e.g., visual feedback, auditory feedback, or haptic feedback), and the input from the user may be received in any form (including acoustic input, speech input, or tactile input).

The systems and technologies described herein may be implemented in a computing system that includes background components (for example, a data server), or a computing system that includes middleware components (for example, an application server), or a computing system that includes front-end components (for example, a user computer with a graphical user interface or a web browser, through which the user may interact with the implementation of the systems and technologies described herein), or include such background components, intermediate computing components, or any combination of front-end components. The components of the system may be interconnected by any form or medium of digital data communication (e.g., a communication network). Examples of communication networks include: a local area network (LAN), a wide area network (WAN), and the Internet.

The computer system may include a client and a server. The client and server are generally remote from each other and interacting through a communication network. The client-server relation is generated by computer programs running on the respective computers and having a client-server relation with each other. The server may be a cloud server, a server of a distributed system, or a server combined with a block-chain.

It should be understood that the various forms of processes illustrated above may be used to reorder, add or delete steps. For example, the steps described in the disclosure could be performed in parallel, sequentially, or in different orders, as long as the desired result of the technical solution disclosed in the disclosure is achieved, which is not limited herein.

The above specific implementations do not constitute a limitation on the protection scope of the disclosure. Those skilled in the art should understand that various modifications, combinations, sub-combinations and substitutions may be made according to design requirements and other factors. Any modification, equivalent replacement and improvement made within the spirit and principle of the disclosure shall be included in the protection scope of the disclosure.

What is claimed is:

1. A method for predicting a structure of a protein complex, comprising:
obtaining an initial coordinate of each amino acid residue in a target protein complex, and obtaining a target residue pair feature, a first multiple sequence alignment (MSA) feature and a second MSA feature of each protein monomer in the target protein complex; and
inputting the initial coordinate of each amino acid residue, and the target residue pair feature, the first MSA feature and the second MSA feature of each protein monomer to an N-level fold iteration network layer, obtaining a target coordinate of each amino acid residue by predicting a torsion angle, a position transformation at residue level and a position transformation at monomer chain level of each amino acid residue via the N-level fold iteration network layer, and obtaining a predicted structure of the protein complex based on the target coordinate of each amino acid residue,
wherein the first MSA feature is an MSA feature subjected to normalization, the second MSA feature is an MSA feature subjected to a mapping process, and N is an integer greater than 1.

2. The method of claim 1, further comprising:
obtaining a target residue code 1 and a candidate position transformation 1 of a first level fold iteration network layer by predicting the position transformation at residue level and the position transformation at monomer chain level for each amino acid residue by inputting the initial coordinate, the target residue pair feature, and the second MSA feature to a first-level fold iteration network layer;
for a m^{th}-level fold iteration network layer, obtaining a target residue code m and a candidate position transformation m of the m^{th}-level fold iteration network layer by predicting the position transformation at residue level and the position transformation at monomer chain level for each amino acid residue by inputting the target residue pair feature, a target residue code m−1 and a candidate position transformation m−1 of a (m−1)^{th}-level fold iteration network layer to the m^{th}-level fold iteration network layer, where m ranges from 2 to N; and
predicting a side chain and a torsion angle for the first MSA feature and a target residue code N of an N^{th}-level fold iteration network layer via the N^{th}-level fold iteration network layer to obtain the torsion angle of each amino acid residue in the side chain, and obtaining the target coordinate of each amino acid residue based on the torsion angle of each amino acid residue in the side chain and a candidate position transformation N of the N^{th}-level fold iteration network layer.

3. The method of claim 2, wherein obtaining the target residue code 1 and the candidate position transformation 1 of the first-level fold iteration network layer by predicting the position transformation at residue level and the position transformation at monomer chain level for each amino acid residue by inputting the initial coordinate, the target residue pair feature, and the second MSA feature into the first-level fold iteration network layer, comprises:
obtaining the target residue code 1 by performing invariant point attention mechanism and a mapping process on the initial coordinate, the target residue pair feature, and the second MSA feature via the first-level fold iteration network layer;
obtaining a first position transformation 1 of the amino acid residue by performing position transformation prediction at residue level on the target residue code 1, and obtaining a second position transformation 1 of the amino acid residue by performing position transformation prediction at monomer chain level on the target residue code 1; and
obtaining the candidate position transformation 1 of the first-level fold iteration network layer by performing position update based on the first position transformation 1, the second position transformation 1 and the initial coordinate.

4. The method of claim 2, wherein obtaining the target residue code m and the candidate position transformation m of the m^{th}-level fold iteration network layer by predicting the position transformation at residue level and the position transformation at monomer chain level for each amino acid residue after inputting the target residue pair feature, the target residue code m−1 and the candidate position transformation m−1 of the (m−1)^{th}-level fold iteration network layer to the m^{th}-level fold iteration network layer, comprises:
obtaining the target residue code m by performing invariant point attention mechanism and a mapping process on the candidate position transformation m−1 and the target residue pair feature of the (m−1)^{th}-level fold iteration network layer inputted to the m^{th}-level fold iteration network layer;
obtaining a first position transformation m of the amino acid residue by performing position transformation prediction at residue level on the target residue code m, and obtaining a second position transformation m of the amino acid residue by performing position transformation prediction at monomer chain level on the target residue code m; and
obtaining the candidate position transformation m of the m^{th}-level fold iteration network layer based on the first position transformation m and the second position transformation m.

5. The method of claim 2, wherein obtaining the first position transformation of each amino acid residue by performing position transformation prediction at residue level on the target residue code of the amino acid residue, comprises:
obtaining the first position transformation of each amino acid residue by performing a mapping processing on the target residue code of each amino acid residue based on a backbone update algorithm.

6. The method of claim 2, wherein obtaining the second position transformation of each amino acid residue by performing position transformation prediction at monomer chain level on the target residue code of the amino acid residue, comprises:
dividing two or more adjacent amino acid residues into different monomer chains based on the target residue code of each amino acid residue; and
for target amino acid residues in each monomer chain, obtaining a candidate residue code at monomer chain level by performing a mean calculation on target residue codes of the target amino acid residues, and obtaining the second position transformation of each amino acid residue in the monomer chain by performing a mapping process on the candidate residue code based on a multi-layer neural network structure.

7. The method of claim 6, wherein the multi-layer neural network structure comprises three layers of linear networks, and obtaining the second position transformation of each amino acid residue in the monomer chain by mapping the candidate residue code based on the multi-layer neural network structure, comprises:
obtaining a first transformation representation by inputting the candidate residue code to a first linear network in the three layers of linear networks for a mapping process;
obtaining a second transformation representation by inputting the first transform representation in the three layers of linear networks to a second linear network for a mapping process; and
obtaining the second position transformation of the amino acid residue in the monomer chain by inputting the first transformation representation and the second transformation representation to a third linear network in the three layers of linear networks for a mapping process.

8. The method of claim 1, wherein obtaining the target residue pair feature of each protein monomer in the target protein complex, comprises:
obtaining a template feature of the protein monomer, and constructing a pairing feature of an amino acid sequence of the protein monomer;
obtaining a candidate residue pair feature by inputting the template feature of the protein monomer to a linear network for a mapping process and adding a mapped template feature and the pairing feature of the protein monomer together; and
obtaining the target residue pair feature of the protein monomer by inputting the candidate residue pair feature to a preset encoder for encoding.

9. The method of claim 8, wherein obtaining the template feature of each protein monomer, comprises:
matching a target amino acid sequence of the protein monomer with a plurality of first amino acid sequences in a protein structure data base respectively to obtain a second amino acid sequence with a similarity greater than a preset threshold; and
determining a distance between coordinates of amino acid residues of the second amino-acid sequence as the template feature of the protein monomer.

10. The method of claim 8, wherein constructing the pairing feature of the amino acid sequence of each protein monomer, comprises:
obtaining candidate sequence code features by inputting the amino acid sequence of the protein monomer into two preset linear networks;
obtaining a first sequence code feature and a second sequence code feature by adding null dimensions to different directions of the candidate sequence code features respectively; and
obtaining the pairing feature of the protein monomer by adding the first sequence code feature and the second sequence code feature.

11. The method of claim 1, wherein obtaining the first MSA feature and the second MSA feature of each protein monomer in the target protein complex, comprises:
querying a homologous sequence of the protein monomer from a plurality of gene sequence data bases based on a target amino acid sequence of the protein monomer;
obtaining a candidate MSA feature of the protein monomer by performing MSA on the homologous sequence of the protein monomer;
obtaining a target MSA feature of the protein monomer by inputting the candidate MSA feature of the protein monomer to a preset encoder for encoding; and
obtaining the first MSA feature of the protein monomer by performing normalization on the target MSA feature of the protein monomer, and obtaining the second MSA feature of the protein monomer by performing a mapping process on the target MSA feature of the protein monomer.

12. An electronic device, comprising:
at least one processor; and
a memory, communicatively connected to the at least one processor,
wherein the memory is configured to store instructions executable by the at least one processor, and the at least one processor is configured to:
obtain an initial coordinate of each amino acid residue in target a protein complex, and obtain a target residue pair feature, a first multiple sequence alignment (MSA) feature and a second MSA feature of each protein monomer in the target protein complex; and
input the initial coordinate of each amino acid residue, and the target residue pair feature, the first MSA feature and the second MSA feature of each protein monomer to an N-level fold iteration network layer, obtain a target coordinate of each amino acid residue by predicting a torsion angle, a position transformation at residue level and a position transformation at monomer chain level of each amino acid residue via the N-level fold iteration network layer, and obtain a predicted structure of the protein complex based on the target coordinate of each amino acid residue,
wherein the first MSA feature is an MSA feature subjected to normalization, the second MSA feature is an MSA feature subjected to a mapping process, and N is an integer greater than 1.

13. The electronic device of claim 12, wherein the at least one processor is further configured to:
obtain a target residue code 1 and a candidate position transformation 1 of a first level fold iteration network layer by predicting the position transformation at residue level and the position transformation at monomer chain level for each amino acid residue by inputting the initial coordinate, the target residue pair feature, and the second MSA feature to a first-level fold iteration network layer;
for a m^{th}-level fold iteration network layer, obtain a target residue code m and a candidate position transformation m of the m^{th}-level fold iteration network layer by predicting the position transformation at residue level and the position transformation at monomer chain level for each amino acid residue by inputting the target residue pair feature, a target residue code m−1 and a candidate position transformation m−1 of a (m−1)^{th}-level fold iteration network layer to the m^{th}-level fold iteration network layer, where m ranges from 2 to N; and
predict a side chain and a torsion angle for the first MSA feature and a target residue code N of an N^{th}-level fold iteration network layer via the N^{th}-level fold iteration network layer to obtain the torsion angle of each amino acid residue in the side chain, and obtain the target coordinate of each amino acid residue based on the torsion angle of each amino acid residue in the side chain and a candidate position transformation N of the N^{th}-level fold iteration network layer.

14. The electronic device of claim 13, wherein the at least one processor is further configured to:
obtain the target residue code 1 by performing invariant point attention mechanism and a mapping process on the initial coordinate, the target residue pair feature, and the second MSA feature via the first-level fold iteration network layer;
obtain a first position transformation 1 of the amino acid residue by performing position transformation prediction at residue level on the target residue code 1, and obtain a second position transformation 1 of the amino acid residue by performing position transformation prediction at monomer chain level on the target residue code 1; and
obtain the candidate position transformation 1 of the first-level fold iteration network layer by performing position update based on the first position transformation 1, the second position transformation 1 and the initial coordinate.

15. The electronic device of claim 13, wherein the at least one processor is further configured to: obtain the target residue code m by performing invariant point attention mechanism and a mapping process on the candidate position transformation m−1 and the target residue pair feature of the (m−1)^{th}-level fold iteration network layer inputted to the m^{th}-level fold iteration network layer;
obtain a first position transformation m of the amino acid residue by performing position transformation prediction at residue level on the target residue code m, and obtain a second position transformation m of the amino acid residue by performing position transformation prediction at monomer chain level on the target residue code m; and
obtain the candidate position transformation m of the m^{th}-level fold iteration network layer based on the first position transformation m and the second position transformation m.

16. The electronic device of claim 13, wherein the at least one processor is further configured to: obtain the first position transformation of each amino acid residue by performing a mapping processing on the target residue code of each amino acid residue based on a backbone update algorithm.

17. The electronic device of claim 13, wherein the at least one processor is further configured to: divide two or more adjacent amino acid residues into different monomer chains based on the target residue code of each amino acid residue; and
for target amino acid residues in each monomer chain, obtain a candidate residue code at monomer chain level by performing a mean calculation on target residue codes of the target amino acid residues, and obtain the second position transformation of each amino acid residue in the monomer chain by performing a mapping process on the candidate residue code based on a multi-layer neural network structure.

18. The electronic device of claim 12, wherein the at least one processor is further configured to: obtain a template feature of the protein monomer, and construct a pairing feature of an amino acid sequence of the protein monomer;
obtain a candidate residue pair feature by inputting the template feature of the protein monomer to a linear network for a mapping process and adding a mapped template feature and the pairing feature of the protein monomer together; and
obtain the target residue pair feature of the protein monomer by inputting the candidate residue pair feature to a preset encoder for encoding.

19. The electronic device of claim 12, wherein the at least one processor is further configured to:
query a homologous sequence of the protein monomer from a plurality of gene sequence data bases based on a target amino acid sequence of the protein monomer;
obtain a candidate MSA feature of the protein monomer by performing MSA on the homologous sequence of the protein monomer;
obtain a target MSA feature of the protein monomer by inputting the candidate MSA feature of the protein monomer to a preset encoder for encoding; and
obtain the first MSA feature of the protein monomer by performing normalization on the target MSA feature of the protein monomer, and obtain the second MSA feature of the protein monomer by performing a mapping process on the target MSA feature of the protein monomer.

20. A non-transitory computer-readable storage medium storing computer instructions, wherein when the computer instructions are executed by a computer, the method predicting the structure of the protein complex is realized, the method comprising:
obtaining an initial coordinate of each amino acid residue in a target protein complex, and obtaining a target residue pair feature, a first multiple sequence alignment (MSA) feature and a second MSA feature of each protein monomer in the target protein complex; and
inputting the initial coordinate of each amino acid residue, and the target residue pair feature, the first MSA feature and the second MSA feature of each protein monomer to an N-level fold iteration network layer, obtaining a target coordinate of each amino acid residue by predicting a torsion angle, a position transformation at residue level and a position transformation at monomer chain level of each amino acid residue via the N-level fold iteration network layer, and obtaining a predicted structure of the protein complex based on the target coordinate of each amino acid residue,
wherein the first MSA feature is an MSA feature subjected to normalization, the second MSA feature is an MSA feature subjected to a mapping process, and N is an integer greater than 1.
